(19) United States
(12) Patent Application Publication
(10) Pub. No.: US 2019/0105344 A1
(43) Pub. Date: Apr. 11, 2019

(54) ORAL MOLECULAR IODINE COMPOSITION AND METHOD

(71) Applicants: Jack KESSLER, Southborough, MA (US); Iogen, LLC, Carlsbad, CA (US)

(72) Inventor: Jack KESSLER, Southborough, MA (US)

(21) Appl. No.: 16/088,989

(22) PCT Filed: Sep. 15, 2017
(86) PCT No.: PCT/US17/51884

§ 371 Date: Sep. 27, 2018

Related U.S. Application Data
(60) Provisional application No. 62/395,665, filed on Sep. 16, 2016.

(51) Int. Cl. A61K 33/18

(52) U.S. Cl. CPC A61K 33/18

(57) ABSTRACT

Oral pharmaceutical compositions comprising iodide, iodate and. arachidonic acid in the presence of other pharmaceutical excipients and methods for preparing and using these compositions.

### CROSS-REFERENCE TO RELATED APPLICATIONS

This application claims priority to U.S. Provisional Patent Application No. 62/395,665, filed Sep. 16, 2016, which is hereby incorporated by reference in its entirety.

### FIELD OF THE INVENTION

Oral pharmaceutical compositions comprising iodide, iodate and arachidonic acid in the presence of other pharmaceutical excipients and methods for preparing and using these compositions.

### BACKGROUND OF THE INVENTION

U.S. Pat. Nos. 5,885,592 and 6,248,335, each of which is herein incorporated by reference, describe methods of generating molecular iodine in the stomach of a mammal using an iodate and iodate. The primary safety concern from chronic daily oral dosing of “iodine” compositions is thyroid related adverse events. Iodide is the only species of iodine that (a) enters the thyroid gland and (b) can directly be correlated with thyroid adverse events.

Orally administered molecular iodine rapidly reacts in the stomach by iodinating lipids, amines and di-sulfide bonds. Once a large molecular weight biomolecule such as mucin is iodinated in the stomach the covalently bound iodine can no longer enter the thyroid and contribute to thyrotoxicity. Therefore, the oral administration of molecular iodine predominantly leads to two categories of iodine species: (1) potentially thyrotoxic iodide anions; and (2) non-toxic organically bound iodine.

Thrall and co-workers demonstrated that a large portion of orally administered molecular iodine is incorporated into lipids and therefore not available to enter the thyroid. See, e.g., Karla Thrall, Ph.D. Thesis, Formation of Organic By-Products Following Consumption of Iodine Disinfected Drinking Water, Summary and Conclusion Section, Oregon State University, Department of Chemistry, 1992; and Sherer et al., Journal of Toxicology and Environmental Health, Vol. 32, pp. 89-101, 1991.

In another study, subchronic administration of iodide to male rats increased their thyroid weight at an iodide concentration of 10 mg/kg; however molecular iodine did not affect thyroid weight even at concentrations of 100 mg/kg. This data indicates that iodide can affect thyroid weight in mammals at concentrations that are 10-fold less than a comparable effect from molecular iodine. Thrall's data demonstrates that once orally administered molecular iodine reacts with organic biomolecules in the stomach the resulting iodinated species does not contribute to thyrotoxicity.

Iodine intake has been correlated to breast health by numerous researchers. See, e.g., Kessler, J. H., The effect of supraphysiologic levels of iodine on patients with cyclic mastalgia. Breast J, 2004. 10(4): pp. 328-336. However, within the past two decades a mechanistic basis has been demonstrated and, for the first time, been supported with experimental data.

The researchers in the laboratory of Carmen Aceves have experimentally identified the intermediate that is likely responsible for the positive association between breast health and iodine intake. This intermedicate is 6-iodo-lipd (6-IL) also referred to as 6-iodo-5-hydroxy-8,11,14, eicosatrienoic acid or 6-iodolactone.

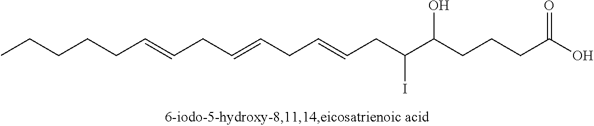 [SMILES: CCCCC/C=C/C/C=C/C/C=C/CC(I)C(O)CCCC(=O)O]

In 2005, Aceves demonstrated that molecular iodine (but not iodide) inhibits the formation of carcinogen induced tumors. See, e.g., Garcia-Solis, P., et al., Inhibition of N-methyl-N-nitrosourea-induced mammary carcinogenesis by molecular iodine (I₂) but not by iodide (I-) treatment: Evidence that I₂ prevents cancer promotion. Mol Cell Endocrinol, 2005. 236(1-2): pp. 49-57.

In 2007, Aceves demonstrated that: (1) the antineoplastic effect of molecular iodine involves formation of 6-IL and peroxisome proliferator-activated receptor gamma (PPARγ); and (2) both molecular iodine and 6-IL induce the same intracellular pathways and is associated with the formation of 6-IL.

In the art, it is well known that cancer cells contain high concentrations of 6-IL which may explain why iodine exerts an antineoplastic effect in these cells. See, e.g., Aceves, et al., Apoptotic and Antiproliferative Effects of Iodine Supplements on Human Breast Cancer Tumors. Thyroid, 2007, 17 (Suppl 1); p. 1; Aceves, et al., 6-Iodo-d-Lactone Identified as Novel Ligand of Peroxisome Proliferator-Activated Receptors (PPARs); and Thyroid, 2007. 17 (Suppl 1): p. 1,; Nava-Villalba and Aceves, 6-lodolactone, key mediator of antitumoral properties of iodine, Prostaglandins and Other Lipid Mediators, 2014, 112, p. 27-33, respectively.

The present invention relates to oral pharmaceutical compositions containing iodide and iodate that are capable of delivering molecular iodine to the stomach of mammals and uses thereof.

### BRIEF DESCRIPTION OF THE DRAWINGS

FIG. 1 illustrates a re-oxidation range with graph and iodide measurement using HPLC as described in Example 3, according to one embodiment of the invention.

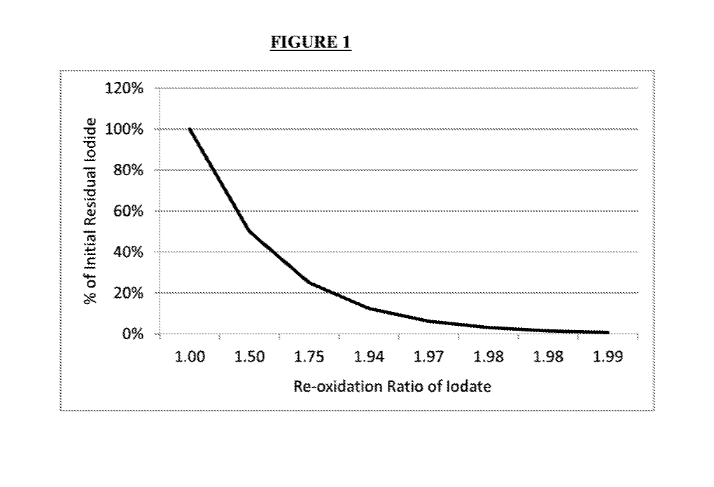

### BRIEF SUMMARY OF THE INVENTION

The application discloses methods and compositions of matter that minimize the ratio of thyrotoxic to nonthyrotoxic iodine formed in the stomach of mammals by the reaction of iodate with iodide. Also disclose here are means to provide for the in situ formation of 6-IL, which is the iodinated organic molecule that has been demonstrated to be a key intermediate responsible for breast health benefits that are associated with chronic daily oral administration of supraphysiologic levels of iodine.

In various embodiment, provided herein is an oral pharmaceutical composition comprising; (a) iodide and iodate in a re-oxidation ratio of between about 1.1 to about 2.0; (b) at least one one pharmaceutically acceptable excipient; and (c) at least one pH control agent. In some of these embodiments, the effective pH of the composition is between about 7.0 and 12.0. In some embodiments, the concentration of iodide and iodate is substantially identical throughout the composition. In yet other embodiments, the iodide and iodate in the composition delivers molecular iodine to the stomach of a subject when administered to the subject.

Also provided herein are pharmaceutical composition comprising: (a) iodide and iodate in a re-oxidation ratio of between about 1.1 to about 2.0, wherein the iodide and iodate in the composition delivers molecular iodine to the stomach of a subject when administered to the subject; (b) arachidonic acid, wherein the molar ratio of arachidonic acid to molecular iodine formed in the stomach is between about 1.1 to about 100: (c) at least one one pharmaceutically acceptable excipient; and (d) at least one pH control agent. In some of these embodiments, the molecular iodine formed in the stomach of the subject reacts with the arachidonic acid to form an iodinated lipid. In other embodiments, the concentration of iodide and iodate is substantially identical throughout the composition. In still other embodiments, the effective pH of the composition is between about 7.0 and 12.0.

In some embodiments of the inventions disclosed herein, the molar ratio of arachidonic acid to molecular iodine is between about 5 to about 90; or the molar ratio of arachidonic acid to molecular iodine is between about 10 to about 80; or the molar ratio of arachidonic acid to molecular iodine is about 50.

In some embodiments of the inventions disclosed herein, the arachidonic acid is present in the composition in an amount between about 100 mgs to about 1,500 mgs; or the arachidonic acid is present in the composition in an amount between about 500 mgs to about 1,500 mgs; or the arachidonic acid is present in the composition in an amount of about 1,200 mgs.

In various embodiments, the subject is a mammal.

In some embodiments, the source of iodide in the composition is calcium iodide, sodium iodide, potassium iodide, magnesium iodide, zinc iodide, cupric iodide, manganese iodide, or a mixture thereof.

In some embodiments, the source of iodate is calcium iodate, sodium iodate, potassium iodate, magnesium iodate, zinc iodate, cupric iodate, manganese iodate, or a mixture thereof.

In various embodiments of the invention described herein, the pH control agent is sodium carbonate, calcium carbonate, potassium carbonate, magnesium carbonate, sodium hydroxide, bentonite (Al₂O₃.4SiO₂.H₂O), dibasic calcium phosphate dihydrate, magnesium oxide, magnesium trisilicate, sodium bicarbonate, dibasic sodium phosphate, tribasic sodium phosphate, dibasic potassium phosphate, tribasic potassium phosphate, or a mixture thereof.

In some embodiments of the invention described herein, the pharmaceutical excipient is sodium algiate, alginic acid, dicalcium phosphate tricalcium group phosphate, microcellulose, citric acid, fructose, magnesium stearate, α-cyclodextrin, β-cyclodextrin, γ-cyclodextrin, povidone, hydroxypropylmethylcellulose, hydroxypropylmethylcellulose phthalate, disodium phosphates, sodium stearate, sorbitol, starch, sucrose, sodium acetate, sodium carboxymethylcellulose, ethyl vanillin, mannitol, sodium chloride, calcium sulfate, maltodextrin, dextrose, dextrin, dextrates, myvatex-TL, saccharin, or a mixtures thereof.

### DETAILED DESCRIPTION OF THE INVENTION

The present invention will be described more fully hereinafter. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Unless otherwise indicated, all publications, patents and patent applications cited herein, whether supra or infra, are hereby incorporated by reference in their entirety. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are the portions of the document which are incorporated by reference herein.

To more readily facilitate an understanding of the invention and its preferred embodiments, the meanings of terms used herein will become apparent from the context of this specification in view of common usage of various terms and the explicit definitions of other terms provided in the glossary below or in the ensuing description.

### Glossary

It should he noted that, as used in this specification, the singular forms “a,” “an,” and “the” include plural referents unless the context clearly dictates otherwise. Thus, for example, reference to a “gelling agent” refers to a single gelling agent as well as to several different gelling agents, reference to an “excipient” includes a single excipient as well as two or more different excipients, and the like.

“Optional” or “optionally” means that the subsequently described circumstance may or may not occur, so that the description includes instances where the circumstance occurs and instances where it does not.

The term “pharmaceutically acceptable” in reference to an entity or ingredient is one that causes no significant adverse toxicological effects in the patient at specified levels, or if levels are not specified, in levels known to be acceptable by those skilled in the art. All ingredients described in this application are pharmaceutically acceptable.

The term “molecular iodine” refers to diatomic iodine, which is represented by the chemical symbol I₂ (CAS Registry Number: 7553-56-2) whether dissolved, suspended or in a solid state. Molecular iodine is also referred to as “elemental iodine” when in the solid state.

The term “iodide” or “iodide anion” refers to the iodide anion, which is represented by the chemical symbol I⁻ (CAS Registry Number: 20461-54-5). Suitable counter-ions for the iodide anion include sodium, potassium, calcium, and the like. Iodide salts such as calcium iodide, sodium iodide, potassium iodide, magnesium iodide, zinc iodide, cupric iodide, and manganese iodide are readily available and are representative of the types of compounds that can, individually or in combination, serve as a suitable source of iodide. In fact, for the purpose of this application “a source of iodide” includes any nontoxic chemical entity that releases iodide anion upon dissolution in water.

For the purposes of this invention, the term “residual iodide” is the iodide anion formed when molecular iodine iodinates a biomolecule in the mammalian stomach leading to the formation of: (1) a covalent bond between iodine and the biomolecule; and (2) a molecule of iodide.

The term “iodate” refers to the iodate anion which carries a negative charge and is represented by the chemical symbol IO₃. Iodate salts such as calcium iodate, sodium iodate, potassium iodate, magnesium iodate, zinc iodate, cupric iodate, and manganese iodate are easily obtained and are representative of the types of entities that can, individually or in combination, serve as a suitable source of iodate. In fact, for the purpose of this application “a source of iodate includes any chemical entity that releases the iodate anion upon dissolution in water.

Non-limiting examples of commonly available salts of iodate that serve as suitable sources for iodate for various embodiments of the invention described herein include, for example, sodium iodate (EC Number: 231-672-5), potassium iodate (EC Number: 231-831-9), and calcium iodate (EC Number: 232-191-3), whether dissolved or in a solid state.

The term “complexed iodine” or “bound iodine” as used herein refers to a mixture of molecular iodine with other chemical species that bind molecular iodine and render the molecular iodine incapable of killing pathogens. Complexing molecule iodine to other chemical species such as iodide and/or polyvinylpyrollidone is a formulation strategy used to increase the stability of molecular iodine. Lugol's solution was the first widely used example of complexed iodine.

The term “thyrotoxic” iodine refers to iodide that is systemically absorbed and can bind with the sodium iodide symporter (NIS) in the thyroid.

The term “ratio of thyrotoxic to nonthyrotoxic iodine” as used herein, refers to the ratio of the amount of iodide divided by the total organically bound iodine from all sources of orally administered iodine.

The term “iodophor” as used herein refers to a mixture of molecular iodine with a polymer(s) that serves to reduce the level of free molecular iodine in solution. Polymers that are used to form iodophors include polyvinylpyrrolidone, copolymers of N-vinyl lactams acrylates and acrylamides, various polyether glycols including nonylphenolethoxylates and the like and combinations thereof. Povidone-iodine (PVP-I) is an iodophor that is most commonly used form of complexed iodine today.

The term “polymer” as used herein includes homopolymers and copolymers and “copolymer” includes a polymer of any length (including oligomers) of two or more types of polymerizable monomers, and therefore includes terpolymers, tetrapolymers, etc., which can include random copolymers, block copolymers, or sequential copolymers.

The term “all iodine species” in a sample refers to the total iodine, irrespective of form, from all iodine containing components within a sample.

The term “ratio of molecular iodine to all iodine species” in a sample refers to the ratio of molecular iodine (I₂) in the sample divided by the concentration of iodine from all iodine species within the sample.

The term “shelf-life” refers to the amount of time that the product can be stored in a suitable package under normal storage conditions and still provide at least 90% of claimed active.

The term “patient” refers to a living organism that can be treated by administration of a suitable embodiment of the teachings contained in this invention.

“Normal storage conditions” in reference to the compositions herein is an environment with a temperature of about 5° C. to about 40° C.; about 10% to about 90% humidity; 1 Atmosphere of Pressure (ATM); and approximately 20% oxygen and 80% nitrogen.

The term “pH control agent” shall refer to chemical(s) that control the effective pH of a composition or a component of a composition. Suitable pH control agents include, but are not limited to, salts of carbonate, phosphate and acetate, formate, succinate, e.g., calcium carbonate, potassium acetate, sodium succinate, and the like.

The terms “single phase” and “dual-phase” refer to packaging configurations anticipated in this application for use with formulations that consist of either a single component or two components. The terra dual-phase also refers to formulations that consist of more than two phases.

The term “re-oxidation ratio” as used herein, refers to the initial molar ratio of iodate to iodide delivered into the stomach of a mammal.

The term “molar ratio of arachidonic acid to molecular iodine” as used herein, is defined as the ratio of (a) the number of moles of arachidonic acid contained in an administered oral dosage divided by (b) the number of moles of molecular iodine formed from the administered oral dosage form by the reaction of of iodate with iodide in the stomach of a mammal.

For the purposes of this invention the term “arachidonic acid” or “AA” shall mean “(5Z,8Z,11Z,14Z)-5,8,11,14-eicosatetraenoic acid which is also known as 5,8,11,14-eicosatetraenoic acid”, which has the chemical structure of:

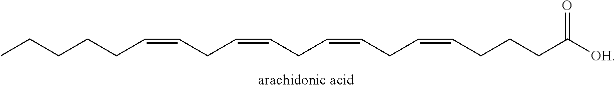 [SMILES: CCCCC/C=C\C/C=C\C/C=C\C/C=C\CCCC(=O)O]

For the purposes of this invention the term “oral pharmaceutical composition of iodide and iodate” shall mean a composition comprised of pharmaceutically acceptable excipients combined with iodide and iodate. Such a composition is to be administered orally to an animal or human. Suitable excipients have been described in the prior art and must not react with either iodide or iodate or with molecular iodine.

It is preferred to uniformly distribute iodate and iodide in the dosage form such that they maintain the correct molar ratio over each dissolution domain in a solid pharmaceutical dosage form. The iodide and iodate are preferably uniformly dispersed. For example, in the case of a solid this can be accomplished by means of a granulation or by “slugging” both iodide and iodate with selected excipients. Common solid pharmaceutical dosage formats such as tablets, capsules or powders are suitable for this invention as is a liquid solution. For the sake of brevity, all patents and other references cited herein are incorporated by reference in their entirety.

The stoichiometry for the oxidation of iodide by iodate under acidic condition is 5:1 with respect to the molar ratio of iodide to iodate. That is, 5 moles of iodide react with 1 mole of iodate to yield 3 moles of molecular iodine. Once formed, a single molecule of molecular iodine will add an atom of iodine to an unsaturated C—C bond of a lipid or other biomolecule an leave a single atom of iodide; iodinated biomolecules cannot be transported into the thyroid and therefore do not contribute to thyrotoxicity. It should be possible to reoxidize this residual iodide anion formed by the reaction of molecule iodine with biomolecules with additional iodate and thereby reduce systemic exposure to the amount of potentially thyrotoxic iodide anions.

Each re-oxidation of residual iodide leads to the formation of additional residual iodide as molecular iodine reacts with organic biomaterial. It is therefore possible to incorporate a defined number of re-oxidation cycles in a formulation. The percentage of the initial residual iodide decreases with each re-oxidatioin cycle; for one (1) cycle this reduction is 50%, for two (2) cycles this reduction is 25%; etc. Five (5) re-oxidation cycles yields 3.75% of the initial residual iodide and by reoxidation cycle eight (8) there is less than 1% of the initial residual iodide. The iodate re oxidation ratio for a stoichiometric reaction is 1; the iodate re-oxidation ratio associated with a residual iodide concentration of less than 1% is about 2.

When females undergo chronic daily supraphysiologic levels of iodine many report increases in breast health. 6-IL has been shown to be a key therapeutic intermediate in the pathway that provides the reported benefits. Therefore, iodination of arachidonic acid in a mammalian stomach by molecular iodine generated via the reaction between iodide and iodate was explored. It was determined that it is possible to iodinate arachidonic by incorporation of arachidonic acid into the dosage form that delivers iodide and iodate.

Arachidoinc acid is an necessary intermediate in inflammatory and cell growth processes. Arachidonic acid supplements are offered commercially from numerous vendors. Daily oral administration of up to 1500 mg have demonstrated no toxicity or safety risk and arachidonic acid is not carcinogenic. Studies in bodybuilders at 1500 mg of arachidonic acid provide evidence that the nutritional agent may reduce systemic inflammation. A scientific advisory from the American Heart Association (AHA) has also provides a favorable evaluation of the health impacts of dietary omega-6 fats, including arachidonic acid. The AHA recommends individuals follow a diet that wherein at least 5-10% of calories are provided by omega-6 fats, including arachidonic acid.

For the purposes of this application arachidonic acid is intended to be incorporated into an oral dosage form such that it is present at the moment the reaction of iodide and iodate form molecular iodine in the stomach. This can be accomplished by means of a granulation or by other means known to those skilled in the art. This serves the purpose of positioning arachidonic acid immediately adjacent to molecular iodine as molecular iodine is formed. The molar ratio of arachidonic acid to molecular iodine contained in the dosage forms contemplated in this application ranges from 1.1 up to 100.

The following examples are illustrative of the teachings of this application and are not meant to limit the invention in any manner.

### EXAMPLES

### Example 1—Iodination of Arachidonic Acid by Molecular Iodine

A stock solution of 10 mg/ml arachidonic acid in ethanol was prepared. Aliquots of this solution were added into vials and the ethanol was removed with a stream of nitrogen followed by vacuum desiccation. A control vial was prepared that had no arachidonic acid.

An iodate/iodide solution was prepared separately by adding NaIO3 (Sigma-Aldrich, St. Louis, Mo.; part number S4007-500G) and NaI (Sigma-Aldrich, St. Louis, Mo.; part number 3831.12-100G) to final concentrations of 3.16 and 20.55 mM, respectively, in 0.64M sodium carbonate in water.

One mL of the iodate/iodide solution was then added to the vials followed by 100 μl 0.5% starch indicator. Two mL of 1N HCl was added and a visual observation of the samples was recorded. Vials were then titrated with 0.01N sodium thiosulfate solution until the samples went from dark blue to colorless.

Since the oxidation of iodide after acidification is diffusion controlled, the reaction of iodate with iodide is essentially instantaneous and five moles of iodide reacts with each mole of iodate to form 3 moles of molecular iodine.

The ratio of iodide to iodate used in this experiment insured that there was unreacted iodate present in the solution after acidification with HCl. The results are shown below in Table 1.

TABLE 1

| Arachidonic Acid | Visual | Thiosulfate | Iodine | Iodine |
|---|---|---|---|---|
| (mg) | Observation | (mL) | (∝mol) | (ppm) |
| 1.0 | Solution | 1.62 | 8.10 | 663 |
| 0.3 | Solution | 1.75 | 8.75 | 716 |
| 0.0 | Solution | 1.86 | 9.30 | 787 |

In the absence of arachidonic acid more molecular iodine is present in the reaction vials. The reduced concentration of molecular iodine indicates that the added arachidonic acid can be iodinated under acidic conditions such as those found in gastric fluid.

### Example 2—Iodination of Sorbic Acid by Moledular Iodine

A stock solution of 13 mg/ml sorbic acid in ethanol was prepared and 77 ul of sorbic acid solution was added to a glass vial and the ethanol was removed with a stream of nitrogen followed by vacuum desiccation. A control vial was prepared that had no sorbic acid.

An iodate/iodide solution was prepared separately by adding sodium iodate (Sigma-Aldrich, St. Louis, Mo.; part number S4007-500G) and sodium iodide (Sigma Aldrich, St. Louis, Mo.; part number 383112-100G) to final concentrations of 3.16 and 20.55 mM, respectively, in 0.64M sodium carbonate in water. One mL of the iodate/iodide solution was then added to the vials followed by 100 μl of 0.5% starch indicator.

A second set of vials had an additional, 188 ∞g of sodium iodate added before the addition of starch indicator. This additional sodium iodate was added to provide additional oxidant to re-oxidize any iodide that might be formed by the iodination of arachidonic acid.

Two mL of 1N HCl was added and a visual observation of the samples was recorded. Vials were then titrated with 0.01N sodium thiosulfate solution until the samples went from dark blue to colorless. The results are shown in Table 2 below.

TABLE 2

| | Sorbic Acid | Additional¹ | Thiosulfate | Iodine | Iodine |
|---|---|---|---|---|---|
| # | (mg) | NaIO₃ (∝g) | (ml) | (∝mol) | (ppm) |
| 1 | 0 | 0 | 1.60 | 8.00 | 677 |
| 2 | 1 | 0 | 1.45 | 7.25 | 613 |
| 3 | 0 | 188 | 1.65 | 8.25 | 698 |
| 4 | 1 | 188 | 1.60 | 8.00 | 677 |

¹In addition to NaIO₃ in iodide/iodate solution (contains 625 mg NaIO₃)

### Example 3—Iodination of Arachidonic Acid by Moledular Iodine

A stock solution of 10 mg/ml arachidonic acid in ethanol was prepared. Aliquots 100 μL of this solution were added into vials and the ethanol was removed with a stream of nitrogen followed by vacuum desiccation. A control vial was prepared that had no arachidonic acid.

An iodate/iodide solution was prepared separately by adding NaIO₃ (Sigma-Aldrich, St. Louis, Mo.; part number S4007-500G) and NaI (Sigma-Aldrich, St. Louis, Mo.; part number 383112-100G) to final concentrations of 3.16 and 20.55 mM, respectively, in 0.64M sodium carbonate in water.

One mL of the iodate/iodide solution was then added to the vials followed by 100 μl of 0.5% starch indicator. A second set of vials had an additional 188 ∞g of NaIO₃ added before the addition of starch indicator. This additional sodium iodate was added to provide additional oxidant to re-oxidize any iodide that might be formed by the iodination of arachidonic acid.

Two mL of 1N HCl was added and a visual Observation of the samples was recorded. Vials were then titrated with 0.01N sodium thiosulfate solution until the samples went from dark blue to colorless. The results are shown in Table 3 below.

TABLE 3

| | Sorbic Acid | Additional¹ | Thiosulfate | Iodine | Iodine |
|---|---|---|---|---|---|
| # | (mg) | NaIO₃ (∝g) | (ml) | (∝mol) | (ppm) |
| 1 | 0 | 0 | 1.90 | 9.50 | 804 |
| 2 | 1 | 0 | 1.70 | 8.50 | 719 |
| 3 | 0 | 188 | 2.20 | 11.00 | 931 |
| 4 | 1 | 188 | 2.10 | 10.50 | 888 |

¹In addition to NaIO₃ in iodide/iodate solution (contains 625 mg NaIO₃)

As shown above, whenever arachidonic acid is present the final concentration of molecular iodine is reduced as compared to no arachidonic acid indicating the iodination of arachidonic acid. Additionally, in the presence of excess iodate oxidant the concentration of molecular iodine formed is higher than when it is omitted indicating that the iodide released during the oxidation of arachidonic acid is re-oxidized.

The theoretical amount of molecular iodine produced with the lower concentration of iodate is 9.48 μmol. The result from Sample 1, i.e. 9.50 μmol, is in agreement with the theoretical amount. One mg (3.28 mol) of arachidonic acid appeared to react with 1.00 mol of molecular iodine (molar ratio of 3.28:1, AA:I₂) as shown in Sample 2.

For sample 3, the amount of iodate was increased from 3.16 μmol to 4.11 μmol by addition of 188 mg of sodium iodate. The results show that 11.00 μmol was produced For sample 4, the addition of 1 mg arachidonic acid should react with 1.00 ∞mol of molecular iodine (viz Sample 1 vs 2). This should produce 0.90 ∞mol of newly created iodide to be available to react with the additional iodate to yield 0.54 ∞mol of molecular iodine. Therefore, 0.54 ∞mol of the molecular iodine lost to arachidonic acid is recovered, so the expected net loss of iodine is 1.00-0.54 or 0.46 ∞mol. This is agreement with the 0.50 ∞mol decrease observed from Sample 3 to Sample 4.

More molecular iodine is generated in the absence of sorbic acid (sample 1 versus 2 and sample 3 versus 4) indicating that molecular iodine can iodinate a saturated bond in sorbic acid. In the presence of additional iodate (sample 2 versus 4) the final concentration of molecular iodine is increased indicating that it is possible to re-oxidize the iodide formed by the iodination of sorbic acid.

### Example 4—Table Generating 6-lodo-lipid

One molecule of iodate is required to oxidize 5 iodide anions to form 3 molecules of I₂: 5I⁻+10₃+6 HCl⁻>3 I₂+3H₂O+6 Cl

When a tablet delivers 3 mg I₂ and the volume of fluid in the stomach is 1 liter, the result is a 1.18E-05 molar solution of I₂. To obtain that concentration of I₂ a molarity of 1.97E-05 iodide and 3.94E-06 iodate (all assuming 100% conversion) is introduced.

Additional experiments are conducted to measure the presence of I₂ in the absence and presence of arachidonic acid in stomach acid.

TABLE 4
Molarities of 3 mg 1₂ in 1 Liter

| | mg | MW | Moles |
|---|---|---|---|
| 12 | 3 | 253.8 | 1.18203E−05 |
| NaI | 2.96 | 149.9 | 1.97465E−05 |
| NaIO₃ | 0.78 | 197.9 | 3.94138E−06 |

Reagents: (1) 0.1N HCL; (2) Sodium carbonate in water at 6.7 grams/100 mL; (3) Sodium iodate 0.98 mg/mL dissolved in the sodium carbonate solution (#2 above).

Add 1 mL of solution 4 into 5 mL of 0.1N HCL. Close with screw top vial and then mix. Remove 1 mL aliquot and titrate using USP method (important to have iodide/starch, i.e. USP method to trap 12). If titration is greater than 330 ppm use 10 mL 0.1N HCL and repeat. Repeat experiment until you can obtain successive titrations that have a CV of 8% or less (n=6). Determine how much arachidonic acid dissolved in solution 2. Add the maximum concentration of soluble arachidonic acid to solution 4 and repeat experiments.

1. An oral pharmaceutical composition comprising:
a. iodide and iodate in a re-oxidation ratio of between about 1.1 to about 2.0;
b. at least one pharmaceutically acceptable excipient; and
c. at least one pH control agent;
wherein the effective pH of the composition is between about 7.0 and 12.0;
wherein the concentration of iodide and iodate is substantially identical throughout the composition;
wherein there is excess iodate remaining after the initial oxidation of substantially all of the iodide in the table; and
wherein the iodide and iodate in the composition delivers molecular iodine to the stomach of a subject when administered to the subject.

2. An oral pharmaceutical composition comprising:
a. iodide and iodate in a re-oxidation ratio of between about 1.1 to about 2.0, wherein the iodide and iodate in the composition delivers molecular iodine to the stomach of a subject when administered to the subject;
b. arachidonic acid, wherein the molar ratio of arachidonic acid to molecular iodine formed in the stomach is between about 1.1 to about 100;
c. at least one pharmaceutically acceptable excipient; and
d. at least one pH control agent;
wherein the molecular iodine formed in the stomach of the subject reacts with the arachidonic acid to form an iodinated lipid;
wherein the concentration of iodide and iodate is substantially identical throughout the composition; and
wherein the effective pH of the composition is between about 7.0 and 12.0.

3. The method of claim 2, wherein molar ratio of arachidonic acid to molecular iodine is between about 5 to about 90.

4. The method of claim 2, wherein molar ratio of arachidonic acid to molecular iodine is between about 10 to about 80.

5. The method of claim 2, wherein molar ratio of arachidonic acid to molecular iodine is about 50.

6. The method of claim 2, wherein the arachidonic acid is present in the composition in an amount between about 100 mgs to about 1,500 mgs.

7. The method of claim 2, wherein the arachidonic acid is present in the composition in an amount between about 500 mgs to about 1,500 mgs.

8. The method of claim 2, wherein the arachidonic acid is present in the composition in an amount of about 1,200 mgs.

9. The method of claim 1, wherein the subject is a mammal.

10. The method of claim 1, wherein the source of iodide is calcium iodide, sodium iodide, potassium iodide, magnesium iodide, zinc iodide, cupric iodide, manganese iodide, or a mixture thereof.

11. The method of claim 1, wherein the source of iodate is calcium iodate, sodium iodate, potassium iodatc, magnesium iodatc, zinc iodatc, cupric iodate, manganese iodate, or a mixture thereof.

12. The method of claim 1, wherein the pH control agent is sodium carbonate, calcium carbonate, potassium carbonate, magnesium carbonate, sodium hydroxide, bentonite (Al₂O₃.4SiO₂.H₂O), dibasic calcium phosphate dihydrate, magnesium oxide, magnesium trisilicate, sodium bicarbonate, dibasic sodium phosphate, tribasic sodium phosphate, dibasic potassium phosphate, tribasic potassium phosphate, or a mixture thereof.

13. The method of claim 1, wherein the pharmaceutical excipient is sodium algiafe, alginic acid, dicalcrum phosphate tri calcium group phosphate, microcellulose, citric acid, fructose, magnesium stearate, α-cyclodextrin, β-cyclodextrin, γ-cyclodextrin, povidone, hydroxypropylmemylcellulosc, hydroxypropyimemylceUulosc phthalate, di sodium phosphates, sodium stearate, sorbitol, starch, sucrose, sodium acetate, sodium carboxyrnethylccHulose, ethyl vanillin, mannitol, sodium chloride, calcium sulfate, maltodextrin, dextrose, dextrin, dextrates, myvatex-TL, saccharin, or a mixtures thereof.
